(19) United States
(12) Patent Application Publication
(10) Pub. No.: US 2025/0176867 A1
(43) Pub. Date: Jun. 5, 2025

(54) ANCHOR ASSEMBLY

(71) Applicant: Unomedical A/S, Lejre (DK)

(72) Inventors: Matthias Herschel, Lejre (DK); Steffen Gyrn, Lejre (DK)

(21) Appl. No.: 19/045,024

(22) Filed: Feb. 4, 2025

Related U.S. Application Data
(63) Continuation of application No. PCT/EP2023/071745, filed on Aug. 4, 2023.
(60) Provisional application No. 63/395,481, filed on Aug. 5, 2022.

(30) Foreign Application Priority Data
Aug. 18, 2022 (GB) 2212069.5

(51) Int. Cl. A61B 5/145; A61B 5/00

(52) U.S. Cl. CPC A61B 5/14532; A61B 5/14503; A61B 5/6832

(57) ABSTRACT

An anchoring assembly for attaching a sensor assembly to a sensor base. The sensor assembly includes an analyte sensing device and a sensor module. The sensor base includes a first surface including a first area adapted to receive the sensor module and an opposite second surface, the second surface including an adhesive. The sensor module includes a sensor module baseplate including an outer surface adapted to be placed on the first surface and an opposite inner surface and a sensor module edge delimiting the outer and the inner surface, a sensor module housing mounted on the inner surface of the sensor module baseplate, and is adapted to be coupled to the first surface of the sensor base by first means.

### TECHNICAL FIELD

The present disclosure generally relates to an anchoring assembly, and more particularly but not exclusively relates to an anchor assembly for attaching a sensor assembly to a sensor base.

### BACKGROUND

Medical components are occasionally attached to one another to form a medical device of the two components. Disclosed herein are systems and methods that may facilitate the connection between two medical device components, and making such connections safe and clear. The subject matter disclosed herein may improve upon existing inserter assemblies for inserting an analytic sensing device, or at least provide a useful alternative to existing technologies.

### SUMMARY

An anchoring assembly according to certain embodiments is configured for attaching a sensor assembly to a sensor base, and generally includes an analyte sensing device and a sensor module. The sensor base includes a first surface including a first area adapted to receive the sensor module and an opposite second surface. The sensor module includes a sensor module baseplate, which includes an outer surface adapted to be placed on the first surface, an opposite inner surface, and a sensor module edge delimiting the outer and the inner surface. The sensor module further includes a sensor module housing mounted on the inner surface of the sensor module baseplate. The sensor module is adapted to be coupled to the first surface of the sensor base by first means. The first means includes first attaching means and guiding means for guiding the sensor module to the first area. Other features and advantages of the disclosed subject matter will become more apparent from the following detailed description taken in conjunction with the accompanying drawings, which illustrate by way of example the principles of the disclosed subject matter.

The invention is defined in the appended claims.

According to an aspect of the invention, there is provided an anchoring assembly for attaching a sensor assembly to a sensor base, the sensor assembly comprising: an analyte sensing device; and a sensor module; wherein the sensor base comprises a first surface comprising a first area adapted to receive the sensor module and an opposite second surface, the second surface comprising an adhesive; wherein the sensor module comprises a sensor module baseplate comprising an outer surface adapted to be placed on the first surface and an opposite inner surface and a sensor module edge delimiting the outer and the inner surface; wherein the sensor module further comprises a sensor module housing mounted on the inner surface of the sensor module baseplate; wherein said sensor module is adapted to be coupled to the first surface of the sensor base by first means; wherein the first means comprises first attaching means being a part of the sensor assembly and adapted to attach to second attaching means being a part of the sensor base; and wherein the first means further comprises guiding means for guiding the sensor module to the first area.

Advantageously, the anchoring assembly of the present invention facilitates the connection between two medical device components (a sensor assembly and a sensor base).

The guiding means may comprise a pater-mater arrangement. The pater part may be a part of the sensor assembly and the mater part may be a part of the sensor base. Alternatively, the pater part may be a part of the sensor base and the mater part may be a part of the sensor assembly.

The guiding means may comprise at least one projecting converging part being a part of either the sensor base or the sensor assembly. Said projecting converging part may be adapted to fit into a projecting-receiving-opening being a part of either the sensor base or the sensor assembly and the part opposite where the projecting converging part(s) is/are placed.

The guiding means may comprise at least two projecting converging parts a first projecting converging part and a second projecting converging part said both projecting converging parts may be placed at the first surface of the sensor module and the sensor assembly may comprise the same amount of projecting-receiving-openings as the number of projecting converging parts said each projecting converging part fits into such an opening.

The guiding means may comprises one projecting converging part being a part of the sensor module base plate. Said sensor module edge of the sensor module base plate may be converging towards the first surface of the sensor module. The guiding means may further comprise a substantially annular wall possibly separated into segments and placed on the first surface. Said projecting converging part may be adapted to snap fit into the annular wall.

The first attachment means may be an integrated part of the guiding means. Said first attaching means may be a projecting converging part being a part of the module plate. Said sensor module edge of the sensor module base plate may be converging towards the first surface of the sensor module. The second attaching means may comprise a substantially annular wall possibly separated into segments. Said projecting converging part may be adapted to snap fit into the annular wall.

The annular wall may comprise a flexible material such as thermoplastic polyethylene TPE. The inside surface of the wall delimiting the first area may be converging in the direction opposite the first area and towards an opening receiving the projecting converging part adapted to snap-fit engagement with the sensor module base plate.

The wall may comprise a second opening in the annular wall adapted to allow an upper part of the continuous glucose monitoring sensor connecting a lower part of the continuous glucose monitoring sensor with the sensor module to pass through the annular wall.

The annular wall may be separated into separate wall-segments. The wall-segments may form a substantially circular opening for receiving the sensor module. The inside surface of the wall delimiting the first area may be converging in the direction opposite the first area and towards an opening receiving the projecting converging part adapted to snap-fit engagement with the substantially circular sensor module base plate.

Each wall segment may comprise flexible latch arms with latch fingers placed at the end of the latch arms module and adapted for snap-fit engagement with the sensor module base plate.

An area between the separate wall-segments may have a larger distance than the distance between the other separate wall-segments forming a second opening in the segmented wall adapted to allow an upper part of the continuous glucose monitoring sensor connecting a lower part of the continuous glucose monitoring sensor with the sensor module to pass through the wall.

The guiding means may be an integrated part of the first and the second attachment means.

The first attaching means may comprise the outer surface. The second attaching means may comprise a double sided adhesive pad one side of the pad adapted to adhere to the first surface of the sensor module and the opposite side of the pad adapted to adhere to the outer surface.

The second attaching means may comprise flexible latch arms with latch fingers and placed on the sensor module and adapted for snap-fit engagement with the first attaching means being a part of the sensor assembly.

The second attaching means may comprise flexible latch arms with latch fingers adapted for snap-fit engagement with the first attaching means comprising the sensor module baseplate.

The second attaching means may comprise flexible latch arms with latch fingers adapted for snap-fit engagement with the first means comprising corrugations placed at the outside of the sensor module housing.

According to another aspect of the invention, there is provided an inserter assembly for inserting an analytic sensoring device such as a continuous glucose monitoring sensor into a subcutaneous tissue of a body, said inserter assembly comprising an insertion device comprising a cover with a first end and an opposite second end and a sensor carrier placed inside the cover comprising insertion means such as an insertion needle, said sensor carrier is moved from a first position to a second position by activating a first energy storing unit activated by an deployment device placed in the insertion device, the inserter assembly further comprising a sensor assembly comprising the continuous glucose monitoring sensor adapted to be inserted and placed into the tissue when the sensor carrier is in the second position; a sensor base placed at the second end of the cover comprising a first surface comprising a first area adapted to receive a sensor module and an opposite second surface, the second surface comprising an adhesive adapted to adhere to a surface wherein the sensor assembly comprises the sensor module connected to the continuous glucose monitoring sensor, said sensor module comprises a sensor module baseplate comprising an outer surface adapted to be placed at the first surface and an opposite inner surface and a sensor module edge delimiting the outer and the inner surfaces; the sensor module further comprises a sensor module housing mounted at the inner surface of the sensor module baseplate, said sensor module is adapted to be moved together with the continuous glucose monitoring sensor by a piston, and in the second position of the sensor carrier the sensor module is by first means adapted to be attached to the first surface of the sensor base and the continuous glucose monitoring sensor is placed in the subcutaneous tissue when the sensor carrier reaches its second position wherein, said first means comprises first attaching means being a part of the sensor assembly and adapted to attach to second attaching means being a part of the sensor base and that the first means further comprises guiding means for guiding the sensor module to the first area.

Advantageously, the present invention facilitates the connection between the sensor module and the sensor base.

With this arrangement, the sensor module becomes attached to the sensor base in the second position. In the first position, the sensor module is detached (spaced from) from the sensor base.

In moving from the first position to the second position, the sensor module moves towards the second end of the cover, i.e. towards the skin.

The guiding means may comprise a pater-mater arrangement. The pater part may be a part of the sensor assembly and the mater part may be a part of the sensor base. Alternatively, the pater part may be a part of the sensor base and the mater part may be a part of the sensor assembly.

The guiding means may comprise at least one projecting converging part being a part of either the sensor base or the sensor assembly. Said projecting converging part may be adapted to fit into a projecting-receiving-opening being a part of either the sensor base or the sensor assembly and the part opposite where the projecting converging part(s) is/are placed.

The guiding means may comprise at least two projecting converging parts a first projecting converging part and a second projecting converging part. Said both projecting converging parts may be placed on the first surface of the sensor module. The sensor assembly may comprise the same amount of projecting-receiving-openings as the number of projecting converging parts said each projecting converging part fits into such an opening.

The guiding means may comprise one projecting converging part being a part of the sensor module base plate. Said sensor module edge of the sensor module base plate may be converging towards the first surface of the sensor module. The guiding means may further comprise a substantially annular wall possibly separated into segments and placed on the first surface. Said projecting converging part may be adapted to snap fit into the annular wall.

The first attachment means may be an integrated part of the guiding means. Said first attaching means may be a projecting converging part being a part of the module plate. Said sensor module edge of the sensor module base plate may be converging towards the first surface of the sensor module. The second attaching means may comprise a substantially annular wall possibly separated into segments. Said projecting converging part may be adapted to snap fit into the annular wall.

The annular wall may comprise a flexible material such as TPE. The inside surface of the wall delimiting the first area may be a coherent surface and may be converging in the direction opposite the first area and towards an opening receiving the projecting converging part adapted to snap-fit engagement with the sensor module base plate.

The wall may comprise a second opening in the annular wall adapted to allow an upper part of the continuous glucose monitoring sensor connecting a lower part of the continuous glucose monitoring sensor with the sensor module to pass through the annular wall.

The annular wall may be separated into separate wall-segments the wall-segments forming a substantially circular opening for receiving the sensor module, the inside surface of the wall delimiting the first area and is converging in the direction opposite the first area and towards an opening receiving the projecting converging part adapted to snap-fit engagement with the substantially circular sensor module base plate.

Each wall segment may comprise flexible latch arms with latch fingers placed at the end of the latch arms module and adapted for snap-fit engagement with the sensor module base plate.

An area between the separate wall-segments may have a larger distance than the distance between the other separate wall-segments forming a second opening in the segmented wall adapted to allow an upper part of the continuous glucose monitoring sensor connecting a lower part of the continuous glucose monitoring sensor with the sensor module to pass through the wall.

The guiding means may be an integrated part of the first and the second attachment means.

The first attaching means may comprise the outer surface and the second attaching means may comprise a double sided adhesive pad. One side of the pad may be adapted to adhere to the first surface of the sensor module and the opposite side of the pad may be adapted to adhere to the outer surface.

The second attaching means may comprise flexible latch arms with latch fingers and placed at the sensor module and adapted for snap-fit engagement with the first attaching means being a part of the sensor assembly.

The second attaching means may comprise flexible latch arms with latch fingers adapted for snap-fit engagement with the first attaching means comprising the sensor module baseplate.

The second attaching means may comprise flexible latch arms with latch fingers adapted for snap-fit engagement with the first means comprising corrugations placed at the outside of the sensor module housing.

The inserter assembly may further comprise a second energy storing unit, said second energy storing unit is activated by activating means when the sensor carrier reaches its second position, by said activating the sensor carrier is adapted to be moved to a third position different from the second position, and in a direction towards the first end of the cover.

The sensor base may be adapted to cover a passage encircled by the second end of the cover, whereby the sensor carrier and the sensor assembly is enclosed in a cavity formed by the cover and the sensor base, and that the inserter assembly comprises means adapted to attach the sensor base to the insertion device.

The first energy storing unit may comprise at least a first spring adapted to bring the sensor carrier from the first position to the second position. The second energy storing unit may comprise a second spring adapted to transport the sensor carrier to the third position. A longitudinal central axis of the first spring may be coinciding with a longitudinal central axis of the second spring.

The first spring may be a coil spring placed inside the piston and surrounding the sensor carrier, said spring is compressed, when the sensor carrier is in the first position, and with a first free end placed nearest the second end of the cover is pressing towards an inside surface of the piston, and the second opposite placed free end of the spring is pressed towards a part of the inserter assembly placed opposite the inside surface of the piston.

The second spring may be a coil spring and placed inside the sensor carrier in a cavity, said first free end of the second spring abuts against an internal base part of the piston and an opposite placed second free end of the second spring abuts against an internal upper surface of the sensor carrier.

An inner housing may encircle the piston, and a base lock is placed around the inner housing and being slidable in the longitudinal direction of the assembly, said inner housing comprises releasable locking means adapted to lock the sensor base to the insertion device when the sensor carrier is in the first position, said locking means is adapted to be released by the base lock when the sensor carrier is in the second position thereby releasing the sensor base from the insertion device.

The base lock may comprise a sleeve surrounding a portion of the inner housing and further comprising flexible arms the longitudinal axes thereof being parallel with a longitudinal axis of the inserter assembly.

The sensor module may comprise electrical connectors for connecting to an electrical circuitry and for transmitting the signals received from the continuous glucose monitoring sensor to a transmitter.

The first surface of the sensor base may comprise the first area adapted to receive the sensor module further comprises a second area separated from the first area comprising a through-going bore in said sensor base. Said through-going bore may be adapted to receive a part of the continuous glucose monitoring sensor and to secure an upper part of the continuous glucose monitoring sensor to the sensor base.

The second area may be adapted to fasten an upper part of the continuous glucose monitoring senor to the sensor base while a lower part of the continuous glucose monitoring sensor comprising a pin-formed elongated body is injected through the bore and is placed in the subcutaneous tissue when the sensor carrier reaches its second position.

The transmitter may comprise an electrical circuitry for transmitting signals received from the sensor assembly to a remote unit and the transmitter further comprises a power supply such as batteries.

The injection means may comprise an insertion needle, which in sectional view is C-formed comprising a lateral opening delimited by sidewalls and a sharp tip and that the lateral opening of the C-form comprises a sharp region at both limiting sidewalls.

The sensor carrier may comprise a needle hub attaching the insertion needle. The needle hub may be configured to be moved by the piston from the first to the second position.

The sensor carrier may comprise a hollow part in which the needle hub is placed and that the second energy storing unit is placed in a circular cavity arranged between the outside of the needle hub and the inside of the hollow part when the sensor carrier is in the first position and the second position.

The piston may comprise a lower piston-surface part adapted to abut against a top-surface of the sensor module, said piston surface and the injection needle are moved synchronous from the first position to the second position, said the injection needle and the lower piston surface part are placed in a distance from each other.

The first means may comprise flexible elongated retention arms comprising retention means such as taps or beards adapted to be locked into recesses.

The activating means may comprise features adapted to cause a rotating movement of the sensor carrier whereby locking means locking the sensor carrier to the piston is released and a force of the second energy storing unit is released moving the sensor carrier from the second position to the third position.

The inserter assembly may comprise a release part comprising a cap with a cap surface placed between the deployment device and the inner housing and a surrounding cap-portion encircling an upper part of the outer surface of the inner housing and further comprising flexible arms locking the deployment device when the sensor carrier is in the first position.

The deployment device may comprise a pressure unit such as a pressure button comprising a first surface-where the pressure is applied to-and a second opposite placed surface region adapted to lock flexible arms placed in the release part-said flexible arms comprises retention units such as beards-towards a lower edge of the deployment device and by a pressure applied to the first surface the retention units are moved in mesial direction and thereby releasing the piston, whereby a force stored in the first energy storing device is released and moving the sensor carrier from the first position to the second position.

The piston may comprise locking means such as pins resting to an upper edge of the release part said locking means are adapted to be released from the release part by a lateral movement of the upper part, which takes place when the retention units are moved radially-inward.

The first energy storing unit may be preloaded with an expansion force F1 and the second energy storing unit may be preloaded with an expansion force F2. Said F1 may be larger than F2.

The piston assembly may comprise the release part, a first energy storing device, an insertion needle fastened to a needle hub, a second energy storing device and a piston.

### BRIEF DESCRIPTION OF THE DRAWINGS

FIG. 1 is a perspective view of an inserter assembly according to certain embodiments.

FIG. 2 is a view of the inside of the inserter assembly shown in FIG. 1.

FIG. 3 is a view of part of the inserter assembly shown in FIG. 1, including a piston, a sensor carrier, a sensor assembly, and a sensor base.

FIG. 4 illustrates certain components of the inserter assembly shown in FIG. 1, including a needle hub, a sensor carrier, a sensor assembly, and a sensor base.

FIG. 5A is an exploded view of a piston assembly.

FIG. 5B is a side and top perspective view of the sensor base comprising a sensor assembly and the sensor base is separated from a transmitter.

FIGS. 6A-C are sectional views of an inserter assembly according to certain embodiments, and illustrates energy storage units and the sensor carrier in a first position, a second position and a third position.

FIG. 7 illustrates an anchoring assembly according to certain embodiments attaching a sensor assembly to a sensor base.

FIG. 8 illustrates the sensor base in FIG. 7, which includes guiding means and attaching means for securing the sensor assembly to the sensor base.

FIG. 9 illustrates the sensor assembly, which is suitable for being secured to the sensor base shown in FIG. 8.

FIG. 10 illustrates an anchoring assembly according to certain embodiments attaching a sensor assembly to a sensor base.

FIG. 11 illustrates the sensor base in FIG. 10, which includes guiding means and attaching means for securing the sensor assembly to the sensor base.

FIG. 12 is a sectional view along the line A-A in FIG. 10; the sensor assembly is that shown in FIG. 9.

FIG. 13 illustrates an anchoring assembly according to certain embodiments attaching a sensor assembly to a sensor base.

FIG. 14 illustrates a sensor assembly according to certain embodiments, which is suitable for being secured to the sensor base shown in FIG. 11.

FIG. 15 is a sectional view along the line B-B in FIG. 13; the sensor assembly is that shown in FIG. 14.

FIG. 16 illustrates an anchoring assembly according to certain embodiments attaching a sensor assembly to a sensor base.

FIG. 17 illustrates the sensor base of FIG. 16, which includes guiding means and attaching means for securing the sensor assembly to the sensor base.

FIG. 18 illustrates a sensor assembly according to certain embodiments, which is suitable for being secured to the sensor base shown in FIG. 17.

FIG. 19 is a sectional view along the line C-C in FIG. 16; the sensor assembly is that shown in FIG. 18.

FIG. 20 is an enlarged view of the part of FIG. 19 that is encircled in D.

FIG. 21 illustrates an anchoring assembly according to certain embodiments attaching a sensor assembly to a sensor base.

FIG. 22 illustrates a sensor assembly according to certain embodiments, which is suitable for being secured to the sensor base shown in FIG. 21.

FIG. 23 illustrates the sensor base of FIG. 21, which includes guiding means and attaching means for securing the sensor assembly to the sensor base.

FIG. 24 is a sectional view along the line E-E in FIG. 21; the sensor assembly is that shown in FIG. 23.

### DETAILED DESCRIPTION

Although the concepts of the present disclosure are susceptible to various modifications and alternative forms, specific embodiments have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should further be appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described.

Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Items listed in the form of “A, B, and/or C” can also mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

In the drawings, some structural or method features may be shown in certain specific arrangements and/or orderings. However, it should be appreciated that such specific arrangements and/or orderings may not necessarily be required. Rather, in some embodiments, such features may be arranged in a different manner and/or order than shown in the illustrative figures unless indicated to the contrary. Additionally, the inclusion of a structural or method feature in a particular figure is not meant to imply that such feature is required in all embodiments and, in some embodiments, may be omitted or may be combined with other features.

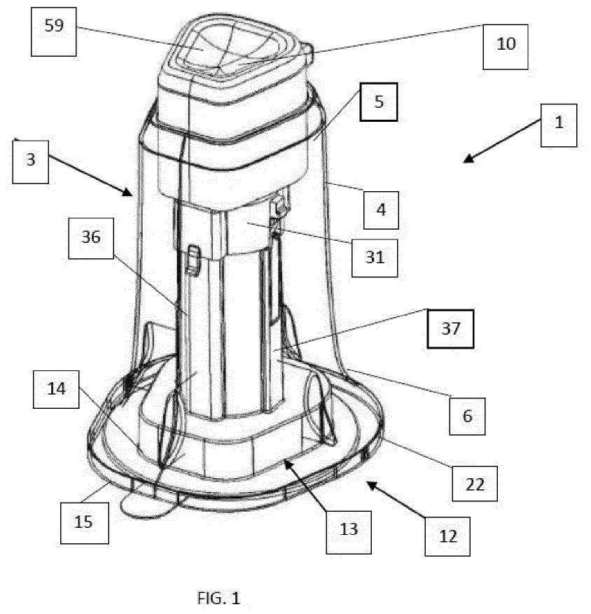

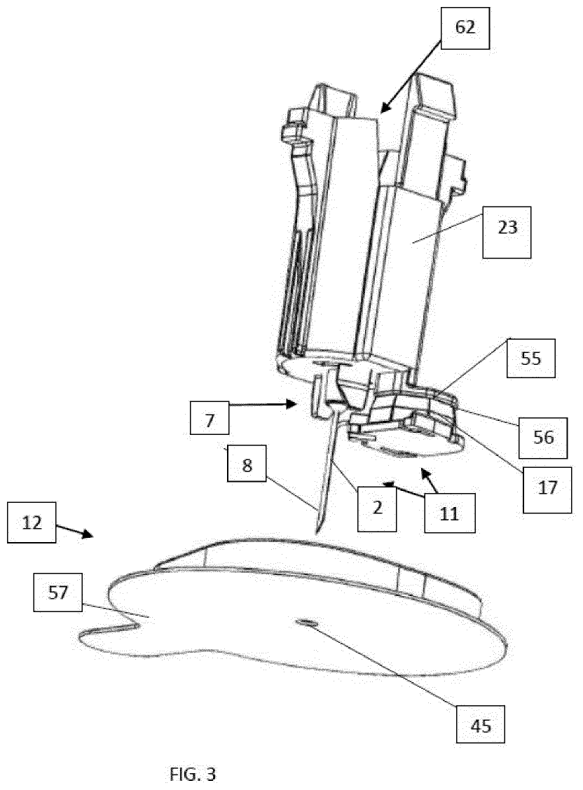

With reference to FIG. 1, illustrated therein is an inserter assembly 1 according to certain embodiments. The inserter assembly 1 is configured for inserting an analytic sensing device, such as a continuous glucose monitoring sensor 2 (FIG. 3) into a subcutaneous tissue of a body. The analytic sensing device could also be a sensor for monitoring the inflammation of the tissue, the pH value of the tissue, the number of the red and/or white corpuscles, and/or one or more additional or alternative parameters. The inserter assembly 1 includes an insertion device 3 comprising a cover 4 with a first end 5 and an opposite second end 6. Further, the inserter assembly 1 includes a sensor base 12 that is placed at the second end 6 of the cover 4, and which includes a base plate 13 with a first surface 14 and an opposite second surface 15. The second surface 15 comprises an adhesive adapted to adhere to a surface.

The sensor base 12 may be releasably connected, or unconnected to the second end 6 of the cover 4. The first surface 14 of the device 10 may have connection points to enable the cover 4 to attach to the sensor base 12.

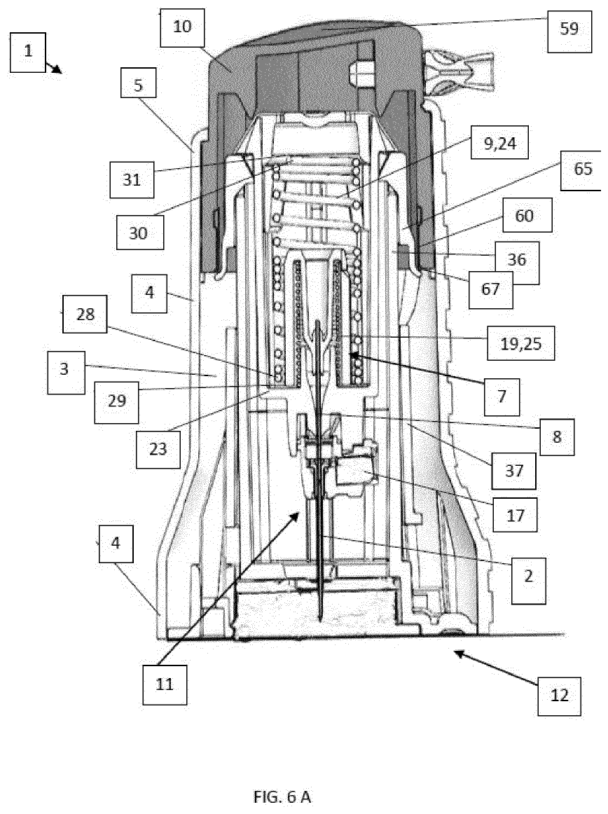

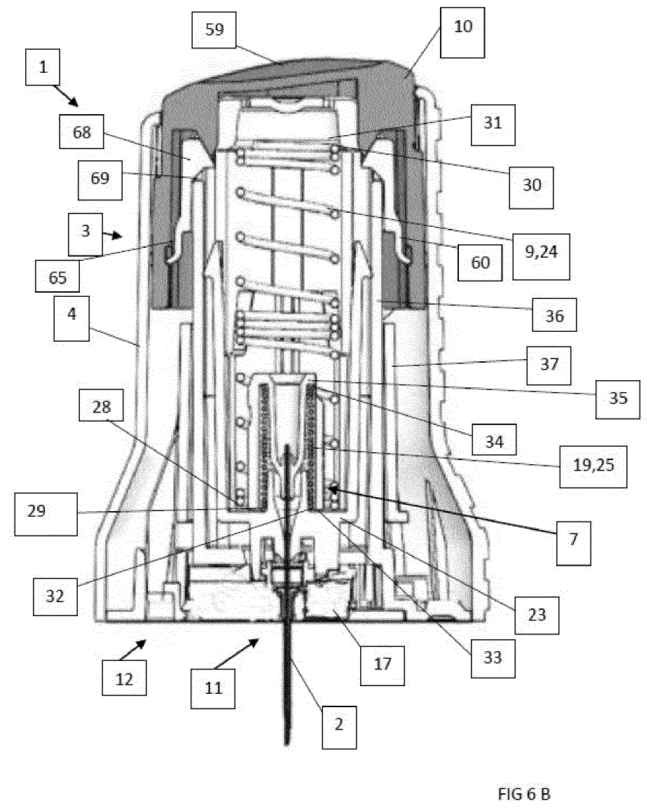

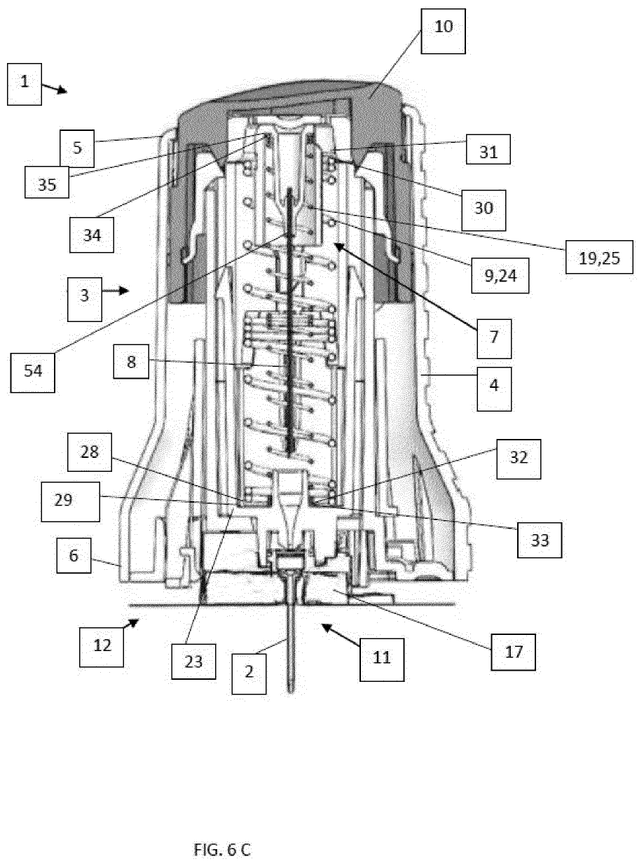

A deployment device 10, which in the illustrated form is provided as a pressure button, is placed in the first end 5 of the cover 6. The deployment device 10 activates a first energy storage unit, and is to be explained with reference to FIGS. 6A-C. A pressure may be applied to the first surface 14 of the deployment device 10 to activate the inserter assembly 1. The cover 6 hides an inner housing 36, which houses a piston, a sensor carrier, a sensor assembly, the first energy storage unit, and a second energy storage unit.

A slidable sleeve called a base lock 37 is placed around the inner housing. This inner housing 36 comprises releasable locking means adapted to lock the sensor base 12 to the insertion device 3 and to be released after reaching a second position by the action of the base lock 37. The functionality will be explained with reference to FIGS. 6A-C. The unlocking takes place by an axial movement of the base lock 37. The axial movement is executed by the piston. The piston includes pins that slide in longitudinal placed recesses in the inner housing and thereby move the base lock 37 slightly in the direction towards the sensor base 12. The base lock 12 comprises flexible arms, the longitudinal axes of which are parallel with the direction of movement. The function of the flexible arms is explained below with reference to FIGS. 6A-C.

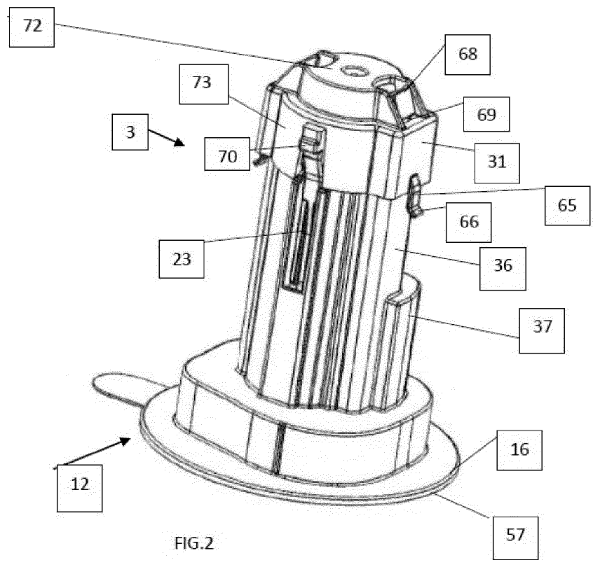

FIG. 2 is a view of the inside of the inserter assembly 1 shown in FIG. 1, where the cover 4 and the deployment assembly 10 are removed to more clearly illustrate the remaining components. FIG. 2 also shows that the sensor base 12 is provided with an adhesive pad 16 at the second surface 15 of the baseplate 13. The adhesive pad 16 may be covered by a removable release liner 57.

A piston 23 is placed inside the inner housing 36 and comprises locking means 68 such as pins, which rest on an upper edge 69 of a release part 31. The release part 31 is a part surrounding the upper portion of the inner housing 36. The release part 31 includes a cap with a cap surface 72 placed between the button and the inner housing, and a surrounding portion 73 encircling an upper portion of the outer surface of the inner housing 36. Flexible arms 65 are placed in the lower portion of the surrounding portion 73. Each of the flexible arms 65 includes retention units such as beards 66 resting toward a lower edge 67 (FIG. 6A) of the deployment device 10 when the device is in a first position. By a pressure applied to the (top) first surface 59, the retention units 66 are moved in a radially-inward or mesial direction (towards the center of the device), and the upper edge 69 of the release part 31 is moved in opposite direction. The piston 23 is thereby released, whereby a force stored in a first energy storage device is released to move the sensor carrier from the first position to the second position.

The piston further comprises radial pointing pins 70 that are placed in the upper part of the piston 23 and which are slidably arranged in a recess of the inner housing 36. By the release of the piston, the pins are moved in the direction towards the sensor base 12. By the movement, the pins 70 push the base lock 37, which releases the locking mechanism between the sensor base 12 and the insertion device 3 as explained with reference to FIGS. 6A-C.

FIG. 3 shows details of the inserter assembly 1 shown in FIG. 1. The inserter assembly 1 generally includes a piston 23, a sensor carrier 7, and a sensor assembly 11. These components are hidden inside the inner housing 36 shown in FIGS. 1 and 2. The sensor assembly 11 comprises an analyte sensing device, such as a continuous glucose monitoring sensor 2, and a sensor module 17. The sensor assembly 11 is attached to the sensor carrier 7, which includes an introducer needle 8. The sensor carrier 7 comprises a cavity 62, which is open in the direction of the pressure button and closed in the opposite direction by a needle hub fastening the introducer needle 8 to the sensor carrier 7. In the cavity 62, a second energy storage unit is placed. The continuous glucose monitoring sensor 2 may be attached to the introducer needle 8.

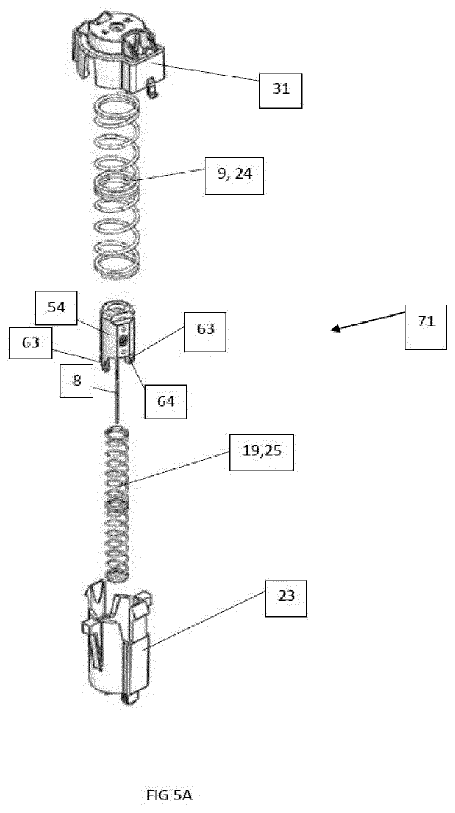

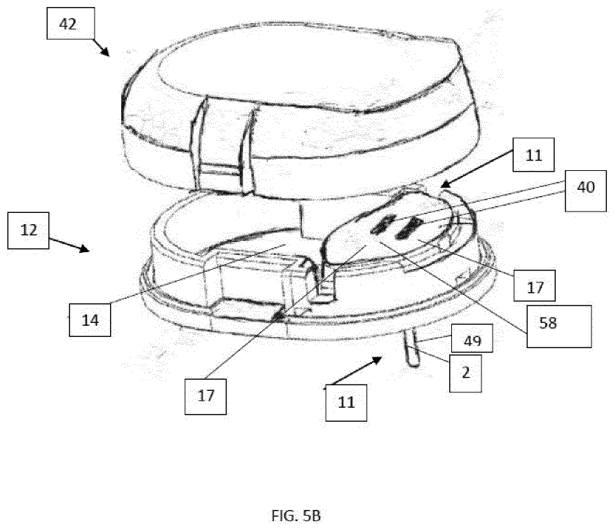

The piston 23 is the part that injects the continuous glucose monitoring sensor 2 into the tissue, and is activated by a spring explained in further detail below. The introducer needle 8 is fastened to the lower part of the piston 23 in such a way that it follows the piston 23 in the direction towards the tissue, but is released from the piston 23 after the continuous glucose monitoring sensor 2 is introduced into the tissue and is moved in opposite direction away from the tissue. That is, (as described further below in relation to FIG. 5A) the introducer needle 8 is connected non-releasably to the needle hub 54, and that unit of hub and needle is located within the piston 23 and moves with the piston 23 towards the tissue. Then, as the needle hub meets the sensor base 12, there is a rotation of the needle hub that causes a second spring to be unlocked and the needle hub and introducer needle 8 to be forced back in the opposite direction.

A top surface 56 of the sensor module 17 abuts, and is releasably connected with a lower piston surface 55. Thereby the introducer needle 8, the continuous glucose monitoring sensor 2, and the sensor module 17 are moved synchronously during the injection of the sensor assembly 11. The top surface 56 of the sensor module and lower piston surface 55 may be connected by a high friction connection (e.g. by virtue of the surface roughness and material properties, such as a silicone membrane), or by a snap lock connection. This allows the respective surfaces to be held together before use, and then released when the sensor module 17 is fitted to the sensor base 12 after deployment. The continuous glucose monitoring sensor 2 is injected through a through-going bore 45 in the sensor base 12, while the sensor module 17 is attached to the first surface 14 of the sensor base 12.

At the end of the insertion step, the sensor module gets released from the inserter device, by release of the high friction connection or snap lock, and attached to the sensor base.

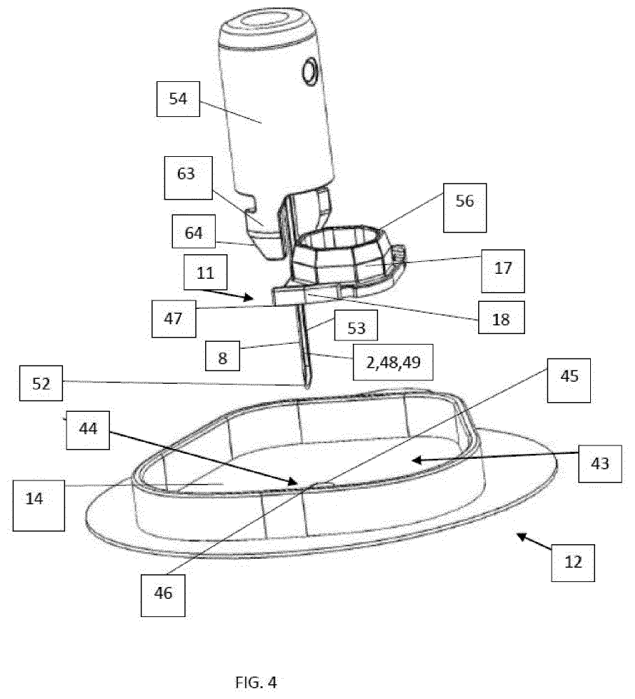

FIG. 4 illustrates certain parts of the inserter assembly 1 shown in FIG. 1, including a needle hub 54, the sensor carrier, the sensor assembly 11, and the sensor base 12. The sensor assembly 11 comprises the sensor module 17 and the continuous glucose monitoring sensor 2. A lower part 48 of the continuous glucose monitoring sensor 2 is formed as an elongated pin-formed body 49. Further, the continuous glucose monitoring sensor 2 comprises an upper part 47 that is connected to the sensor module 17. In the example, the continuous glucose monitoring sensor 2 is not placed within the sensor module 17, but is instead placed laterally in relation to the sensor module 17, and is connected to the sensor module 17 by the upper part 47 of the continuous glucose monitoring sensor 2. The connecting part may be bent by about 90° (e.g., between 85° and) 95°. The sensor 2 may be connected to the sensor module 17 by a wire, for example.

The continuous glucose monitoring sensor 2 is connected to the introducer needle 8, e.g. positioned within the introducer needle 8 or at the tip of the introducer needle 8. The introducer needle 8 of the sensor carrier 7 has a C-shaped cross-section, and includes a sharp tip 52 and a lateral opening defining the opening in the C shape. This opening may include a sharp region 53 delimiting the opening. The introducer needle 8 is opposite the sharp tip 52 connected to the needle hub 54, and which is moved downwards by the piston 23.

The first surface 14 of the sensor base 12 comprises a first area 43 adapted to receive the sensor module 17. The first surface 14 of the sensor base 12 further comprises a second area 44 separate from the first area 43 comprising the through-going bore 45. The through-going bore 45 is adapted to receive a part of the continuous glucose monitoring sensor 2. The continuous glucose monitoring sensor 2 is injected through the bore 45 into the tissue. The continuous glucose monitoring sensor 2 is caught and secured to the sensor base 12 by third means 46. The third means 46 may be constructed as a conical part surrounding and keeping the continuous glucose monitoring sensor 2 in place by press fitting. The third means 46 may also be formed as flexible arms that catch grooves placed in a body surrounding the continuous glucose monitoring sensor 2.

The sensor module 17 may be fastened to the sensor base 12 in the first area 43 by taps or flexible arms 18, which may be clipped into recesses constructed in the sensor base 12. The construction may also be the opposite, i.e. having taps or flexible arms constructed in the sensor base 12 that are clipped into recesses in the sensor module 17. Other fastening mechanisms may be used. For example, the sensor module 17 may be fastened by adhesive or by a push fit or wedge type of arrangement.

When the sensor assembly 11 is placed in the sensor base 12, the sensor carrier 7 may be automatically withdrawn, thereby leaving the continuous glucose monitoring sensor 2 in the tissue. The sensor carrier 7 is withdrawn from the sensor assembly 11 by the activating of a second energy storage unit, which may take the form of a coiled spring. The second energy storage unit is placed within the sensor carrier 7. A first energy storage unit, by which the introducer needle 8 carrying the sensor assembly 11 injects the continuous glucose monitoring sensor 2 into the tissue, is placed around the sensor carrier 7 but within the piston 23. Each of the energy storage units may, for example, take the form of preloaded springs.

FIG. 5A is an exploded view of a piston assembly 71 comprising a release part 31, a first energy storage unit 9, an introducer needle 8 fastened to a needle hub 54, a second energy storage unit 19, and the piston 23. The needle hub 54 is initially located within, and hooked into the piston 23 to move together. The lower part of the needle hub 54 comprises two elongated arms 63, each having a free end comprising a slanted edge 64. When the sensor carrier 7 is moved to the second position, the slanted edges 64 hit a corresponding surface of the sensor base 12. Hitting the sensor base 12 stops the travel of the piston 23. That is, the needle hub 54 (including needle 8), at the slanted edges 64 of the arms 63, contacts a correspondingly shaped portion of the sensor base 12, to cause rotation of the needle hub. So, by this interaction, the sensor carrier 7 (including the needle hub and insertion needle) is turned (rotated about 15 to 20 degrees) and released from the sensor assembly 11. The sensor carrier 7 is thereby moved away inside the piston, leaving the piston in the lower position. The needle hub rotation unhooks the hub from the piston. The second spring 19 is also unlocked and pushes the needle hub away from the skin.

FIG. 5B is a side and top perspective view of the sensor base 12 comprising a sensor assembly 11, and a transmitter 42 separated from the sensor base 12. In other words, the insertion device 3 has been removed from the sensor base 12. The transmitter 42 is secured to the sensor base 12 by locking means such as snap means. It comprises the continuous glucose monitoring sensor 2. The elongated pin-formed body 49 is injected into the tissue, while the upper part of the continuous glucose monitoring sensor 2 is secured to the sensor base and to the sensor module 17. The sensor module 17 is secured to the first surface 14 by first means 18 (see FIG. 4) such as flexible arms attaching to recesses placed in the first surface 14. The sensor module 17 has on its top-surface 58—the surface pointing away from the first surface 14—electrical connectors 40. Those electrical connectors 40 are connected to electrical connectors placed in the transmitter 42 when the transmitter 42 is attached and placed on the base part 12. The transmitter 42 comprises an electrical circuitry for transmitting signals received from the sensor assembly 11 to a remote unit. The transmitter 42 further comprises a power supply such as batteries.

FIGS. 6A-C are sectional views of an inserter assembly 1 according to certain embodiments, and illustrate the principle of energy storage units. In these Figures, the sensor carrier 7 is illustrated in a first position (FIG. 6A), a second position (FIG. 6B) and a third position (FIG. 6C). The first position is before the continuous glucose monitoring sensor 2 is injected into the tissue and the device is in a secured and locked state. The second position is when the continuous glucose monitoring sensor 2 is injected into the tissue but before the sensor carrier 7 is withdrawn. The third position is when the sensor carrier 7 is withdrawn and the continuous glucose monitoring sensor 2 is placed in the tissue. The principle of the insertion device 3 is explained below with reference to the three figures. This principle is also the injection principle for the embodiment shown in the figures explained above.

The insertion device 3 comprises a first energy storage unit 9 and a second energy storage unit 19. While other forms are contemplated in the illustrated embodiment, the first energy storage unit 9 is provided in the form of a first spring 24, and the second energy storage unit 19 is provided in the form of a second spring 25. Both springs 24, 25 are cylindrical coil springs, and a longitudinal central axis of the first spring 24 is coaxial with a longitudinal central axis of the second spring 25.

With the sensor carrier 7 in the first position, the first spring 24 is located within the hollow piston 23, which in turn is located inside the inner housing 36. The first spring 24 surrounds the sensor carrier 7. The second spring 25 is located inside the cavity of the hollow sensor carrier 7. The first spring 24 is preloaded with a first spring force F1 (e.g., about 12 N), and the second spring is preloaded with a second spring force F2. The first spring force F1 may be greater than the second spring force F2, and in certain embodiments may be at least twice the second spring force. For example, the first spring force F1 may be about 12 N, and the second spring force F2 may be about 5.5 N. Both preloaded springs may be locked before use.

In the first position, the sensor module 17 is spaced from (detached from) the first surface 14 of the sensor base 12.

When the deployment device 10 is activated by pressing at the first surface 59, the first spring 24 is released. At or around the same time, a flexible fastening means, which secures the piston 23 to the inner housing 36 in the first position, is deactivated by the movement of the deployment device 10 (e.g., the button) in a downward direction. The flexible fastening means may comprise the locking means 68 that are removed from the resting position towards the upper edge 69 of the release part 31 by the applied pressure. The release part 31 is placed immovably in relation to the inner housing 36.

The pressure button 10 comprises the first surface 59, to which the pressure is applied, and a second opposite surface region 60 adapted to lock the flexible arms 65 towards the outside of the piston 23. By the pressure applied to the first surface 59, the flexible arms 60 are moved in the mesial direction and thereby release the piston 23 as the beards/taps are moved in the opposite direction. The force F1 stored in the first energy storing device 9 is released, and moves the sensor carrier 7 from the first position to the second position.

The first spring 24 is in a compressed state when the sensor carrier 7 is in the first position. A first free end 28 of the first spring 24 is placed nearest the second end 6 of the cover 4, and presses toward an inside surface 29 of the piston 23. The second opposite placed free end 30 of the first spring 24 presses towards an opposite placed part that is an inner top surface of the release part 31.

The base lock 37 is positioned around a lower part of the inner housing 36, and is slightly slidable in the longitudinal direction towards the sensor base 12. In the illustrated form, the base lock 37 is a substantially cylindrical unit and comprises longitudinal flexible arms, the longitudinal axes of which may be parallel with the longitudinal axis of the insertion device 3. Each of the flexible arms comprises one or more beards 66. The insertion device and the sensor base are locked to each other by pins/tabs in one part interacting with recesses in the other part. When the base lock 37 is moved during the action from the first position to the second position and reaches the second position, the flexible arms and the beards thereon interact with the locking and removes the pins/tabs from the recesses. As a result, the inserter device and the sensor base are released from each other. This may be done after the sensor carrier has released the sensor assembly 11.

When the sensor carrier 7 reaches the second position, the sensor assembly 11 is released from the introducer needle 8 and the sensor carrier 7, as the sensor carrier 7 reaches a surface at the sensor base 12, thereby forcing the sensor carrier 7 to rotate. By the rotation caused by these activating means, the senor assembly 11 is released, whereby the force F2 of the second spring 25 is released and the second spring 25 is expanded, thereby bringing the sensor carrier 7 into its third position. The second spring 25 is located inside the sensor carrier 7 in a cavity and a first free end 32 of the second spring 25 abuts against an internal base part 33 of the piston 23. The opposite second free end 34 of the second spring 25 abuts an upper surface 35 of the sensor carrier 7. When the second spring 25 is released as the sensor carrier is released from the sensor assembly, the sensor carrier 17 is moved in the direction opposite the injection direction, thereby reaching the third position.

The sensor base 12 is released from the inserter device 3 by the deactivating of the locking mechanism of the base lock 37. This is done by the flexible arms of the base lock 37, which are pressed to the sides and thereby miss the engagement to the recesses in the base part 12. When the locking mechanism is deactivated, the rotating movement of the sensor carrier 17 takes place. As a result of this rotational movement, locking means locking the sensor carrier to the piston are released, and the force of the second energy storage unit F2 is thereby released. As the base part 12 comprises an adhesive pad 16 placed at the second surface 15, the sensor base 12 is now fastened to the skin surface.

Preferably, the insertion needle of the sensor carrier is in sectional view C-shaped, and comprises a lateral opening delimited by sidewalls and a sharp tip. In certain embodiments, the lateral opening of the C-shape comprises a sharp region at both limiting sidewalls.

The insertion needle could also be a solid needle such as it is common known from the medical injection devices. In that case, the continuous glucose monitoring sensor may be formed hollow surrounding the insertion needle. The continuous glucose monitoring sensor could also be formed C-shape and partly surround the insertion needle.

Additionally or alternatively, the sensor could be formed as an injection needle comprising a sharp tip and in some cases a sharp sidewall. Thereby it is not relevant with a separate insertion needle as the sensor itself ensures that the continuous glucose monitoring sensor is injected properly into the tissue.

With the above described arrangement, an inserter assembly is provided for inserting an analyte sensing device into the skin, moveable between a first position (before insertion), a second position (sensor injected) and a third position (sensor carrier withdrawn). The device is arranged such that deployment of the button 10 causes an axial movement of a piston 23 and sensor carrier towards a sensor base 12. A sensor 2 is injected into tissue and a sensor module 17 is attached to the sensor base 12. The sensor carrier is retracted, and the insertion device may be removed to leave the sensor base 12 attached to the skin. A transmitter 42 may be connected on the sensor base 12.

A sensor assembly engages with a sensor base only as the analyte sensor is implanted into the skin, i.e. only as the sensor module moves to the second position.

A transmitter may be connected to a sensor base (including sensor assembly) after the sensor assembly has been attached to the skin. In this way, the transmitter does not need to be attached to the skin as part of the sensor assembly. This gives more design freedom to the inserter assembly, enabling a simple construction. In addition, the transmitter may be interchanged if required, without changing the whole sensor assembly.

Furthermore, the construction of the inserter assembly enables a compact arrangement of the features to enable injection of the sensor 2 into a subcutaneous tissue.

In order to secure the placement of the sensor module into subcutaneous tissue, with the above arrangement, the configuration and the timing of the release and retraction of the insertion needle and rotation do not damage the sensor.

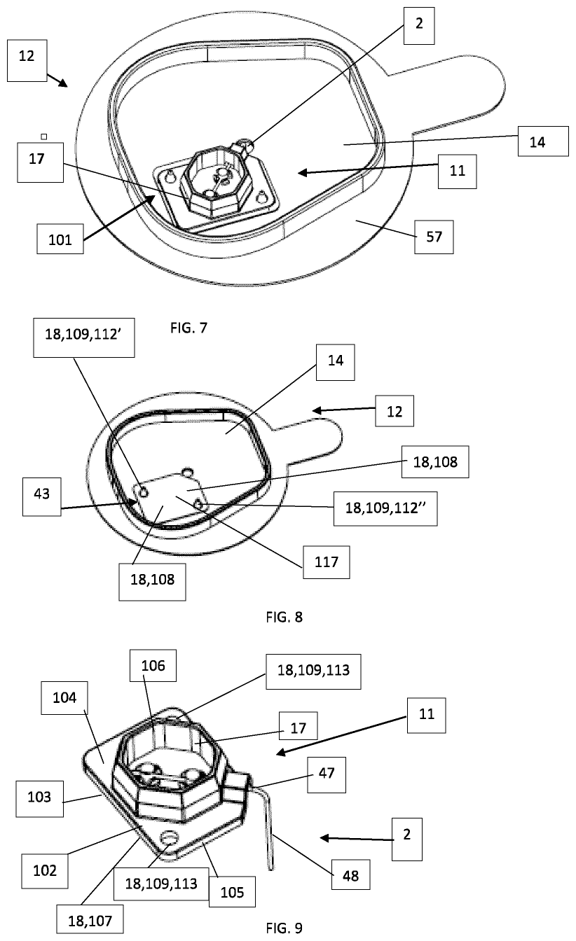

A first embodiment of an anchoring assembly 101 for attaching the sensor assembly 11 to the sensor base 12 according to certain embodiments will be explained with reference to FIGS. 7-9. FIG. 7 shows the sensor assembly 11 attached to the sensor base 12, which may be done by a first embodiment of an anchoring assembly 101. FIG. 8 shows the sensor base 12 before the sensor assembly 11 is attached, the sensor base 12 including first means 18. The first means 18 comprises second attaching means 108 and guiding means 109. FIG. 9 shows an example of a sensor assembly 11 suitable for being attached to the sensor base 12 shown in FIG. 8 (and FIG. 11). The sensor assembly 11 includes first means 18, which in turn comprise first attaching means 107 and guiding means 109.

The sensor base 12 includes the first surface 14 that includes the first area 43, which is adapted to receive the sensor module 17. A release liner 57 may cover the second surface. The sensor module 17 comprises a sensor module baseplate 102, which includes an outer surface 103 adapted to be placed at the first surface 14 and an opposite inner surface 104. Both surfaces are substantially planar, and may be delimited by a sensor module edge 105. The sensor module 17 further comprises a sensor module housing 106.

The structure of the guiding means 109, which includes projecting converging parts and openings, will now be explained. The guiding means comprises a male-female or pater-mater arrangement. The mater part is a part of the sensor assembly 11, and the pater part is a part of the sensor base 12.

Two projecting converging parts are placed in the first area 43 of the sensor base 12: a first projecting converging part 112′ and a second projecting converging part 112″. The two projecting converging parts are placed with a distance between them. The projecting converging parts 112′, 112″ guide the sensor assembly 11 when placed on the sensor base 12. The projecting converging parts are positioned such that they are caught by two through-going projecting receiving openings 109, 113, which are formed in the sensor module baseplate 102. A doublet adhesive pad 117 is also placed in the first area 43. One side of the pad 117 adheres to the first surface 14, and the other side of the adhesive pad 117 adheres to the outer surface 103 of the sensor module base plate 102 when the sensor assembly 11 is injected in place as explained above. In this way, first attaching means 107 comprises the outer surface 103, and the second attaching means 108 comprises the double sided adhesive pad 117. One side of the pad 117 is adapted to adhere to the first surface 14 of the sensor module, and the opposite side of the pad 117 is adapted to adhere to the outer surface 103.

The continuous glucose monitoring sensor 2 is injected through the opening provided in the sensor base 12. The continuous glucose monitoring sensor 2 comprises the upper part 47, which is substantially horizontal and is connected to the sensor module 17, and the lower part 48, which is substantially vertical and adapted to be placed in the tissue.

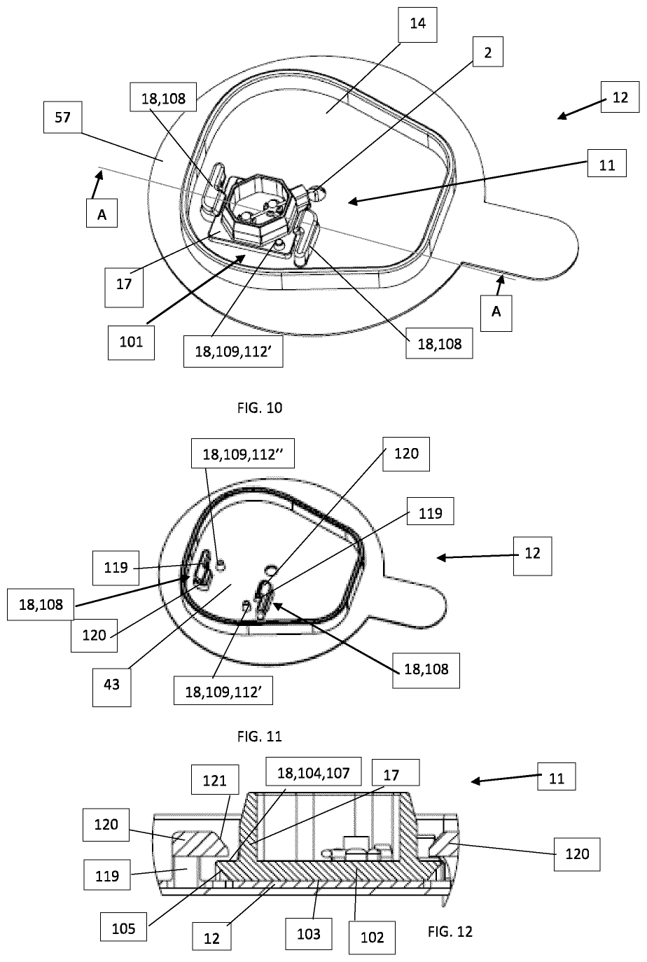

With reference to FIGS. 10-12, illustrated therein is a second embodiment of an anchoring assembly 101 for attaching the sensor assembly 11 to the sensor base 12. The sensor assembly 11 attached to the sensor base 12 is the one shown in FIG. 9. The guiding means is the same as explained with reference to FIGS. 7-9.

The sensor base 12 includes the first surface 14 including the first area 43, which is adapted to receive the sensor module 17. The sensor module 17 comprises a sensor module baseplate 102 comprising an outer surface 103 adapted to be placed at the first surface 14 and an opposite inner surface 104. Both surfaces are substantially planar, and are delimited by a sensor module edge 105 which is slanted and converges towards the outer surface 103. On the sensor base 12, guiding means 109 formed as two projecting converging parts 112′ 112″ are placed in the first area. The projecting converging parts include a first projecting converging part 112′ and a second projecting converging part 112″ as explained above. The projecting converging parts guide the sensor assembly 11 when placed at the sensor base, and are a part of the first means 18. The projecting converging parts are positioned such that they catch the two openings 109, 113 placed in the sensor module baseplate 102.

The first means 18 comprises first attaching means 107 and second attaching means 108. In the illustrated form, the second attaching means 108 is an integrated part of the sensor base 12 and comprises flexible latch arms 119 ending with latch fingers 120. An edge of the latch fingers is slanted such that when the sensor assembly 11 is placed at the sensor base 12, the slanted edge 105 of the sensor module presses towards the slanted surface of the latch fingers 120. Due to the flexibility of the latch arms 119, the fingers 120 are pressed away, and the sensor module 17 is pressed towards the first surface 14. The fingers 120 may then flex back to resting position, and the underside of the latch fingers 120 rest towards a part of the inner surface 104 including the first attaching means 107. By the interaction of the first attaching means 107 and the second attaching means 108, the sensor assembly 11 is safely attached to the sensor base 12 while the guiding means (the projecting converging parts 112′, 112″, interacting with openings in the sensor module base plate 102) ensures that the sensor assembly 11 is placed correctly in the first area 43.

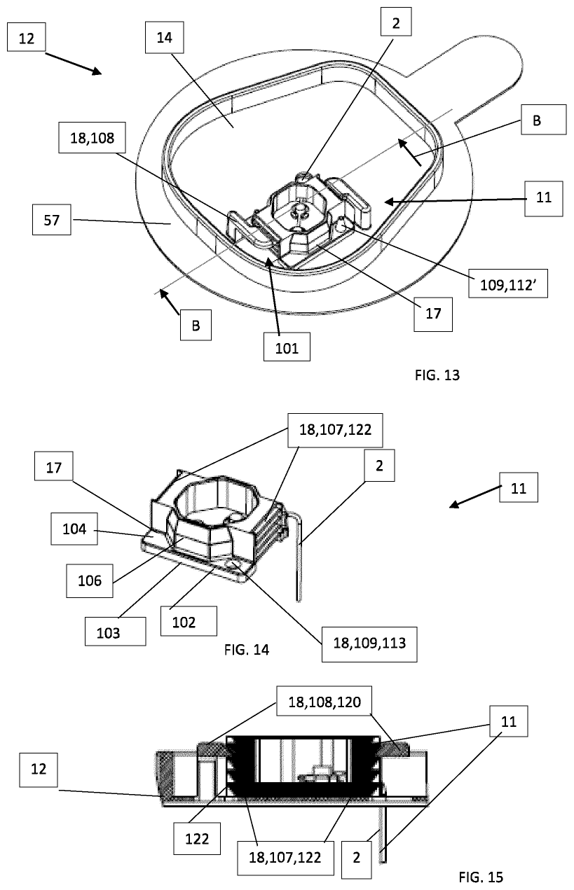

With reference to FIGS. 13-15, illustrated therein is a third embodiment of an anchoring assembly 101 for attaching the sensor assembly 11 to the sensor base 12. The sensor base 12 is equivalent to the one shown in FIG. 11. One difference between this embodiment and the embodiment shown in FIGS. 10-12 is the construction of the sensor assembly 11. The sensor base is constructed as explained with reference to FIGS. 10-12, and includes the same guiding means 109 and the same second attaching means 108.

The sensor module 17 includes a sensor module baseplate 102 comprising an outer surface 103 adapted to be placed at the first surface 14, and an opposite inner surface 104. Both surfaces are substantially planar, and are delimited by a sensor module edge 105. The sensor module 17 further comprises a senor module housing 106. The sensor module housing 106 is constructed with corrugations placed at the outer surface of the housing 106. In this example, the outer surface comprises two plan corrugated wall parts 122 placed opposite each other.

When the sensor assembly 11 is injected in place as explained above, the latch arms 119 (FIG. 11) are moved in lateral direction allowing the sensor assembly 11 to be placed at the sensor base 12. The latch fingers 120 press toward the corrugation and ensure that the sensor assembly 11 is safely fixed to the sensor base 12 due to the press fitting and the friction between the latch fingers and the corrugations. The guiding takes place in the same way as explained with reference to FIGS. 7-9 and FIGS. 10-12.

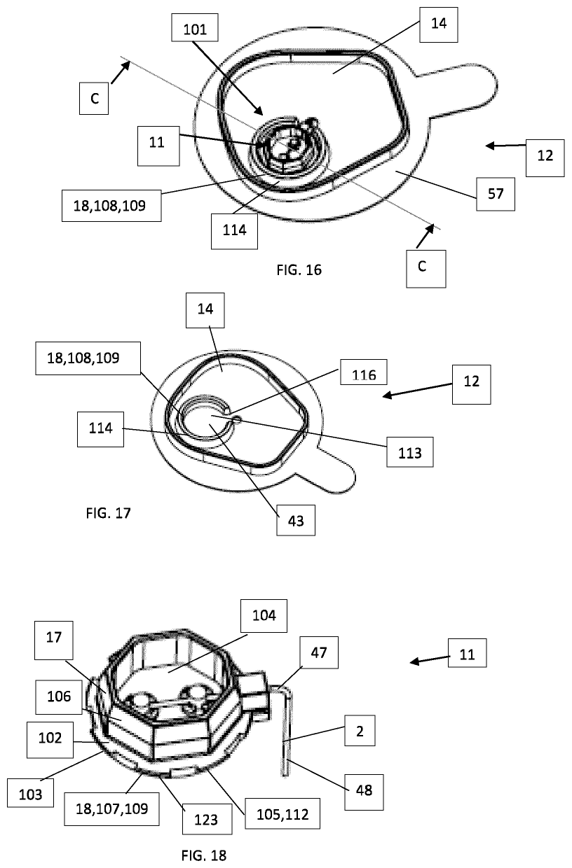

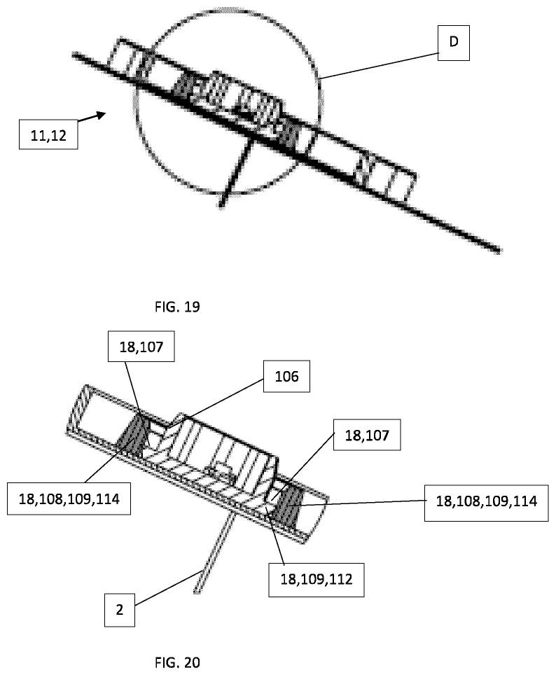

With reference to FIG. 16-20, illustrated therein is a fourth embodiment of an anchoring assembly 101 for attaching the sensor assembly 11 to the sensor base 12. The sensor base 12 comprises the first surface 14 including the first area 43, which is adapted to receive the sensor module 17. A release liner 57 may cover the second surface. The sensor module 17 comprises a sensor module baseplate 102, which includes an outer surface 103 adapted to be placed at the first surface 14 and an opposite inner surface. Both surfaces are substantially planar, and are delimited by a sensor module edge 105. The sensor module 17 further includes a sensor module housing 106.

Now, the structure of the guiding means 109 comprising a projecting converging part and opening will be explained. The guiding means comprises a pater-mater arrangement. The pater part is a part of the sensor assembly 11 and the mater part is a part of the sensor base 12. The guiding means 109 comprises one projecting converging part, which is a part of the sensor module base plate 102. The edge 105 of the sensor module base plate 102 converges towards the first surface 14 of the sensor module 12, and is substantially circular. The edge 105 may be formed with parts (e.g., small tongues 123) that extend in the radial direction, thereby increasing the friction between the sensor assembly 11 and the second attaching means 108 when the sensor assembly is placed on the first surface 14.

The guiding means 109 further comprises a substantially annular wall 114, and when placed on the first surface, the projecting converging part is adapted to snap fit into the annular wall 114. The inside of the annular wall 114 converges in the direction opposite the first surface 14. The annular wall 114 is placed on the first surface 14 such that it forms a substantially circular top-opening 113 (a projecting converging part receiving opening) for receiving the sensor module 17.

The annular wall is formed in a flexible material such as TPE (thermoplastic elastomer). The inside surface of the wall delimits the first area 43, and converges in the direction opposite the first area and towards an opening 113 that receives the projecting converging part and/or the sensor module base plate 102. In this way, the first attachment means 107 and the second attachment means 108 are an integrated part of the guiding means 109, with the first attachment means 107 being the slanted edge 105 comprising the tongues 123, and the second attachment means 108 being the substantially annular flexible wall 114. The wall 114 is not a closed wall, as it comprises a second opening 116 adapted to allow the upper part 47 of the continuous glucose monitoring sensor 2 to pass through the wall 114.

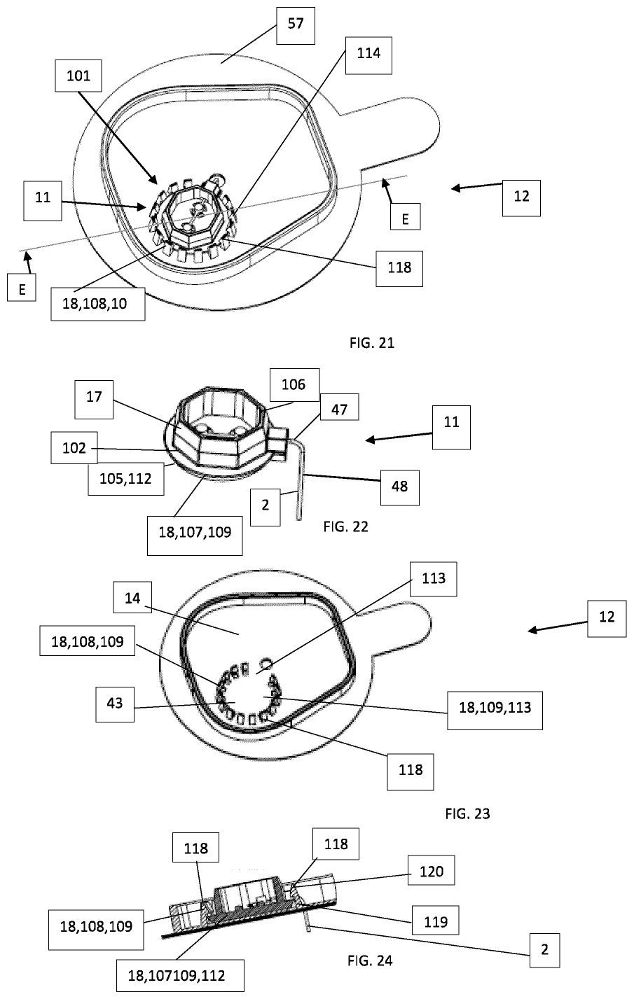

With reference to FIG. 21-24, illustrated therein is a fifth embodiment of an anchoring assembly 101 for attaching the sensor assembly 11 to the sensor base 12. The sensor base 12 comprises the first surface 14 including the first area 43, which is adapted to receive the sensor module 17. A release liner 57 may cover the second surface. The sensor module 17 comprises a sensor module baseplate 102 comprising an outer surface 103 adapted to be placed at the first surface 14 and an opposite inner surface. Both surfaces are substantially planar, and are delimited by a sensor module edge 105. In the illustrated form, the edge is smooth and circular. The sensor module 17 further comprises a senor module housing 106.

The guiding means 109 comprises a pater-mater arrangement. The pater part is a part of the sensor assembly 11 and the mater part is a part of the sensor base 12. The guiding means 109 comprises one projecting converging part, which is a part of the sensor module base plate 102. The edge 105 of the sensor module base plate 102 converges towards the first surface 14 of the sensor module 12. The guiding means 109 further comprises a substantially annular wall 114 placed on the first surface 14. The projecting converging part is adapted to snap fit into the annular wall. The inside of the annular wall 114 converges in the direction opposite the first surface 14. In the illustrated form, the annular wall 114 is separated into separate and discrete wall segments 118. The wall segments 118 are placed on the first surface such that they form a substantially circular top-opening 113 for receiving the sensor module 17.

The first attachment means 107 is an integrated part of the guiding means as the first attaching means 107 is the structure of the sensor module base plate. The edge 105 of the sensor module base plate 102 converges towards the first surface of the sensor module. The second attaching means 108 comprises the substantially annular wall 114, which is separated into wall segments 118. The sensor module base plate 102 snap fits into the annular wall 114

Each wall segment 118 of the annular wall 114 comprises flexible latch arms 119 with latch fingers 120 placed at the end of the latch arms 119 and adapted for snap-fit engagement with the sensor module base plate. The fingers 120 are resting at the inner surface near the edge 105 of the sensor module base plate 102.

While the invention has been illustrated and described in detail in the drawings and foregoing description, the same is to be considered as illustrative and not restrictive in character, it being understood that only the preferred embodiments have been shown and described and that all changes and modifications that come within the spirit of the inventions are desired to be protected.

It should be understood that while the use of words such as preferable, preferably, preferred or more preferred utilized in the description above indicate that the feature so described may be more desirable, it nonetheless may not be necessary and embodiments lacking the same may be contemplated as within the scope of the invention, the scope being defined by the claims that follow. In reading the claims, it is intended that when words such as “a,” “an,” “at least one,” or “at least one portion” are used there is no intention to limit the claim to only one item unless specifically stated to the contrary in the claim. When the language “at least a portion” and/or “a portion” is used the item can include a portion and/or the entire item unless specifically stated to the contrary.

1. An anchoring assembly for attaching a sensor assembly to a sensor base, the sensor assembly comprising:
an analyte sensing device; and
a sensor module;
wherein the sensor base comprises a first surface comprising a first area adapted to receive the sensor module and an opposite second surface, the second surface comprising an adhesive;
wherein the sensor module comprises a sensor module baseplate comprising an outer surface adapted to be placed on the first surface and an opposite inner surface and a sensor module edge delimiting the outer and the inner surface;
wherein the sensor module further comprises a sensor module housing mounted on the inner surface of the sensor module baseplate;
wherein said sensor module is adapted to be coupled to the first surface of the sensor base by first means;
wherein the first means comprises first attaching means being a part of the sensor assembly and adapted to attach to second attaching means being a part of the sensor base; and
wherein the first means further comprises guiding means for guiding the sensor module to the first area.

2. An anchoring assembly according to claim 1, wherein the guiding means comprises a pater-mater arrangement the pater part is a part of the sensor assembly and the mater part is a part of the sensor base or the pater part is a part of the sensor base and the mater part is a part of the sensor assembly.

3. An anchoring assembly according to claim 1, wherein the guiding means comprises at least one projecting converging part being a part of either the sensor base or the sensor assembly; said projecting converging part is adapted to fit into a projecting-receiving-opening being a part of either the sensor base or the sensor assembly and the part opposite where the projecting converging part(s) is/are placed,
or wherein the guiding means comprises at least two projecting converging parts a first projecting converging part and a second projecting converging part said both projecting converging parts are placed at the first surface of the sensor module and that sensor assembly comprises the same amount of projecting-receiving-openings as the number of projecting converging parts said each projecting converging part fits into such an opening,
or wherein the guiding means comprises one projecting converging part being a part of the sensor module base plate, said sensor module edge of the sensor module base plate is converging towards the first surface of the sensor module; and that the guiding means further comprises a substantially annular wall possibly separated into segments and placed on the first surface said projecting converging part is adapted to snap fit into the annular wall.

4. An anchoring assembly according to claim 1, wherein the first attachment means is an integrated part of the guiding means said first attaching means is a projecting converging part being a part of the module plate, said sensor module edge of the sensor module base plate is converging towards the first surface of the sensor module; and that the second attaching means comprises a substantially annular wall possibly separated into segments said projecting converging part is adapted to snap fit into the annular wall, and optionally
(i) wherein the annular wall comprises a flexible material such as thermoplastic polyethylene TPE and the inside surface of the wall delimiting the first area is converging in the direction opposite the first area and towards an opening receiving the projecting converging part adapted to snap-fit engagement with the sensor module base plate, and/or
(ii) wherein the wall comprises a second opening in the annular wall adapted to allow an upper part of the continuous glucose monitoring sensor connecting a lower part of the continuous glucose monitoring sensor with the sensor module to pass through the annular wall.

5. An anchoring assembly according to claim 4, wherein the annular wall is separated into separate wall-segments, the wall-segments forming a substantially circular opening for receiving the sensor module, the inside surface of the wall delimiting the first area is converging in the direction opposite the first area and towards an opening receiving the projecting converging part adapted to snap-fit engagement with the substantially circular sensor module base plate, and optionally
(i) wherein each wall segment comprises flexible latch arms with latch fingers placed at the end of the latch arms module and adapted for snap-fit engagement with the sensor module base plate, and/or
(ii) wherein an area between the separate wall-segments has a larger distance than the distance between the other separate wall-segments forming a second opening in the segmented wall adapted to allow an upper part of the continuous glucose monitoring sensor connecting a lower part of the continuous glucose monitoring sensor with the sensor module to pass through the wall.

6. An anchoring assembly according to claim 1, wherein the guiding means is an integrated part of the first and the second attachment means, and/or
wherein the first attaching means comprises the outer surface and the second attaching means comprises a double sided adhesive pad one side of the pad adapted to adhere to the first surface of the sensor module and the opposite side of the pad adapted to adhere to the outer surface, and/or
wherein the second attaching means comprises flexible latch arms with latch fingers and placed on the sensor module and adapted for snap-fit engagement with the first attaching means being a part of the sensor assembly, or
wherein the second attaching means comprises flexible latch arms with latch fingers adapted for snap-fit engagement with the first attaching means comprising the sensor module baseplate, or
wherein the second attaching means comprises flexible latch arms with latch fingers adapted for snap-fit engagement with the first means comprising corrugations placed at the outside of the sensor module housing.

7. An inserter assembly for inserting an analytic sensoring device such as a continuous glucose monitoring sensor into a subcutaneous tissue of a body, said inserter assembly comprising an insertion device comprising a cover with a first end and an opposite second end and a sensor carrier placed inside the cover comprising insertion means such as an insertion needle, said sensor carrier is moved from a first position to a second position by activating a first energy storing unit activated by an deployment device placed in the insertion device, the inserter assembly further comprising a sensor assembly comprising the continuous glucose monitoring sensor adapted to be inserted and placed into the tissue when the sensor carrier is in the second position; a sensor base placed at the second end of the cover comprising a first surface comprising a first area adapted to receive a sensor module and an opposite second surface, the second surface comprising an adhesive adapted to adhere to a surface wherein the sensor assembly comprises the sensor module connected to the continuous glucose monitoring sensor, said sensor module comprises a sensor module baseplate comprising an outer surface adapted to be placed at the first surface and an opposite inner surface and a sensor module edge delimiting the outer and the inner surfaces; the sensor module further comprises a sensor module housing mounted at the inner surface of the sensor module baseplate, said sensor module is adapted to be moved together with the continuous glucose monitoring sensor by a piston, and in the second position of the sensor carrier the sensor module is by first means adapted to be attached to the first surface of the sensor base and the continuous glucose monitoring sensor is placed in the subcutaneous tissue when the sensor carrier reaches its second position wherein, said first means comprises first attaching means being a part of the sensor assembly and adapted to attach to second attaching means being a part of the sensor base and that the first means further comprises guiding means for guiding the sensor module to the first area.

8. An inserter assembly according to claim 7, wherein the guiding means comprises a pater-mater arrangement the pater part is a part of the sensor assembly and the mater part is a part of the sensor base or the pater part is a part of the sensor base and the mater part is a part of the sensor assembly.

9. An inserter assembly according to claim 7, wherein the guiding means comprises at least one projecting converging part being a part of either the sensor base or the sensor assembly; said projecting converging part is adapted to fit into a projecting-receiving-opening being a part of either the sensor base or the sensor assembly and the part opposite where the projecting converging part(s) is/are placed, or
wherein the guiding means comprises at least two projecting converging parts a first projecting converging part and a second projecting converging part said both projecting converging parts are placed on the first surface of the sensor module, and that sensor assembly comprises the same amount of projecting-receiving-openings as the number of projecting converging parts said each projecting converging part fits into such an opening, or
wherein the guiding means comprises one projecting converging part being a part of the sensor module base plate, said sensor module edge of the sensor module base plate is converging towards the first surface of the sensor module; and that the guiding means further comprises a substantially annular wall possibly separated into segments and placed on the first surface said projecting converging part is adapted to snap fit into the annular wall.

10. An inserter assembly according to claim 7, wherein the first attachment means is an integrated part of the guiding means said first attaching means is a projecting converging part being a part of the module plate, said sensor module edge of the sensor module base plate is converging towards the first surface of the sensor module; and that the second attaching means comprises a substantially annular wall possibly separated into segments said projecting converging part is adapted to snap fit into the annular wall.

11. An inserter assembly according to claim 10, wherein the annular wall comprises a flexible material such as TPE and the inside surface of the wall delimiting the first area is a coherent surface and is converging in the direction opposite the first area and towards an opening receiving the projecting converging part adapted to snap-fit engagement with the sensor module base plate, and/or
wherein the wall comprises a second opening in the annular wall adapted to allow an upper part of the continuous glucose monitoring sensor connecting a lower part of the continuous glucose monitoring sensor with the sensor module to pass through the annular wall.

12. An inserter assembly according to claim 10, wherein the annular wall is separated into separate wall-segments the wall-segments forming a substantially circular opening for receiving the sensor module, the inside surface of the wall delimiting the first area and is converging in the direction opposite the first area and towards an opening receiving the projecting converging part adapted to snap-fit engagement with the substantially circular sensor module base plate, and optionally
wherein each wall segment comprises flexible latch arms with latch fingers placed at the end of the latch arms module and adapted for snap-fit engagement with the sensor module base plate.

13. An inserter assembly according to claim 12, wherein an area between the separate wall-segments has a larger distance than the distance between the other separate wall-segments forming a second opening in the segmented wall adapted to allow an upper part of the continuous glucose monitoring sensor connecting a lower part of the continuous glucose monitoring sensor with the sensor module to pass through the wall.

14. An inserter assembly according to claim 7, wherein the guiding means is an integrated part of the first and the second attachment means, and/or
wherein first attaching means comprises the outer surface and the second attaching means comprises a double sided adhesive pad one side of the pad adapted to adhere to the first surface of the sensor module and the opposite side of the pad adapted to adhere to the outer surface, or
wherein the second attaching means comprises flexible latch arms with latch fingers and placed at the sensor module and adapted for snap-fit engagement with the first attaching means being a part of the sensor assembly, or
wherein the second attaching means comprises flexible latch arms with latch fingers adapted for snap-fit engagement with the first attaching means comprising the sensor module baseplate, or
wherein the second attaching means comprises flexible latch arms with latch fingers adapted for snap-fit engagement with the first means comprising corrugations placed at the outside of the sensor module housing, and/or
wherein the sensor base is adapted to cover a passage encircled by the second end of the cover, whereby the sensor carrier and the sensor assembly is enclosed in a cavity formed by the cover and the sensor base, and that the inserter assembly comprises means adapted to attach the sensor base to the insertion device.

15. An inserter assembly according to claim 7, wherein the inserter assembly further comprises a second energy storing unit, said second energy storing unit is activated by activating means when the sensor carrier reaches its second position, by said activating the sensor carrier is adapted to be moved to a third position different from the second position, and in a direction towards the first end of the cover.

16. An inserter assembly according to claim 15, wherein the first energy storing unit comprises at least a first spring adapted to bring the sensor carrier from the first position to the second position and the second energy storing unit comprises a second spring adapted to transport the sensor carrier to the third position, and a longitudinal central axis of the first spring is coinciding with a longitudinal central axis of the second spring, and optionally
(i) wherein the first spring is a coil spring placed inside the piston and surrounding the sensor carrier, said spring is compressed, when the sensor carrier is in the first position, and with a first free end placed nearest the second end of the cover is pressing towards an inside surface of the piston, and the second opposite placed free end of the spring is pressed towards a part of the inserter assembly placed opposite the inside surface of the piston, or
(ii) wherein the second spring is a coil spring and placed inside the sensor carrier in a cavity, said first free end of the second spring abuts against an internal base part of the piston and an opposite placed second free end of the second spring abuts against an internal upper surface of the sensor carrier.

17. An inserter assembly according to claim 7, wherein an inner housing encircles the piston, and a base lock is placed around the inner housing and being slidable in the longitudinal direction of the assembly, said inner housing comprises releasable locking means adapted to lock the sensor base to the insertion device when the sensor carrier is in the first position, said locking means is adapted to be released by the base lock when the sensor carrier is in the second position thereby releasing the sensor base from the insertion device, and optionally
wherein the base lock comprises a sleeve surrounding a portion of the inner housing and further comprising flexible arms the longitudinal axes thereof being parallel with a longitudinal axis of the inserter assembly.

18. An inserter assembly according to claim 7, wherein the sensor module comprises electrical connectors for connecting to an electrical circuitry and for transmitting the signals received from the continuous glucose monitoring sensor to a transmitter, and optionally
wherein the transmitter comprises an electrical circuitry for transmitting signals received from the sensor assembly to a remote unit and the transmitter further comprises a power supply such as batteries.

19. An inserter assembly according to claim 7, wherein the first surface of the sensor base comprising the first area adapted to receive the sensor module further comprises a second area separated from the first area comprising a through-going bore in said sensor base, said through-going bore is adapted to receive a part of the continuous glucose monitoring sensor and to secure an upper part of the continuous glucose monitoring sensor to the sensor base, and optionally
wherein the second area further is adapted to fasten an upper part of the continuous glucose monitoring senor to the sensor base while a lower part of the continuous glucose monitoring sensor comprising a pin-formed elongated body is injected through the bore and is placed in the subcutaneous tissue when the sensor carrier reaches its second position.

20. An inserter assembly according to claim 7, wherein the injection means comprises an insertion needle, which in sectional view is C-formed comprising a lateral opening delimited by sidewalls and a sharp tip and that the lateral opening of the C-form comprises a sharp region at both limiting sidewalls, and/or
wherein the piston comprises a lower piston-surface part adapted to abut against a top-surface of the sensor module, said piston surface and the injection needle are moved synchronous from the first position to the second position, said the injection needle and the lower piston surface part are placed in a distance from each other.

21. An inserter assembly according to claim 7, wherein the sensor carrier comprises a needle hub attaching the insertion needle; the needle hub is configured to be moved by the piston from the first to the second position, and optionally
wherein the sensor carrier comprises a hollow part in which the needle hub is placed and that the second energy storing unit is placed in a circular cavity arranged between the outside of the needle hub and the inside of the hollow part when the sensor carrier is in the first position and the second position.

22. An inserter assembly according to claim 7, wherein the first means comprises flexible elongated retention arms comprising retention means such as taps or beards adapted to be locked into recesses.

23. An inserter assembly according to claim 15, wherein the activating means comprises features adapted to cause a rotating movement of the sensor carrier whereby locking means locking the sensor carrier to the piston is released and a force of the second energy storing unit is released moving the sensor carrier from the second position to the third position, and/or
wherein the first energy storing unit is preloaded with an expansion force F1 and the second energy storing unit is preloaded with an expansion force F2 said F1 being larger than F2.

24. An inserter assembly according to claim 22, wherein the inserter assembly comprises a release part comprising a cap with a cap surface placed between the deployment device and the inner housing and a surrounding cap-portion encircling an upper part of the outer surface of the inner housing and further comprising flexible arms locking the deployment device when the sensor carrier is in the first position.

25. An inserter assembly according to claim 24,
(i) wherein the deployment device comprises a pressure unit such as a pressure button comprising a first surface-where the pressure is applied to-and a second opposite placed surface region adapted to lock flexible arms placed in the release part-said flexible arms comprises retention units such as beards-towards a lower edge of the deployment device and by a pressure applied to the first surface the retention units are moved in mesial direction and thereby releasing the piston, whereby a force stored in the first energy storing device is released and moving the sensor carrier from the first position to the second position, and optionally
wherein the piston comprises locking means such as pins resting to an upper edge of the release part said locking means are adapted to be released from the release part by a lateral movement of the upper part, which takes place when the retention units are moved radially-inward, or
(ii) wherein a piston assembly comprises the release part, a first energy storing device, an insertion needle fastened to a needle hub, a second energy storing device and a piston.
